(19) United States
(12) Patent Application Publication
(10) Pub. No.: US 2009/0246758 A1
(43) Pub. Date: Oct. 1, 2009

(54) Peptide nucleic acid probes for analysis of microorganisms

(75) Inventors: Mark Fiandaca, Princeton, MA (US); Henrik Stender, Gentofte (DK)

Correspondence Address: EDWARDS ANGELL PALMER & DODGE LLP, P.O. BOX 55874, BOSTON, MA, 02205, US

(73) Assignee: AdvanDx, Inc., Wobum, MA (US)

(21) Appl. No.: 11/921,131

(22) PCT Filed: Jun. 2, 2006
(86) PCT No.: PCT/US06/21614

§ 371 Date: Feb. 17, 2009

Related U.S. Application Data
(60) Provisional application No. 60/687,178, filed on Jun. 2, 2005.
(60) Provisional application No. 60/704,552, filed on Aug. 1, 2005.

(51) Int. Cl. C12Q 1/68; C07H 21/00

(52) U.S. Cl. 435/6; 536/24.32

(57) ABSTRACT

The instant invention provides PNA probes for detection, identification and/or quantitation of microorganisms, e.g., Escherichia coli, Klebsiella pneumoniae, Klebsiella oxytoca, Streptococcus agalactiae, fungi, and Acinetobacter species. The invention further provides methods of using the PNA probes of the invention and kits containing the PNA probes of the invention.

### RELATED APPLICATIONS

This application claims the benefit of U.S. Provisional Application No. 60/687,178, filed Jun. 2, 2005 and U.S. Provisional Application No. 60/704,552, filed Aug. 1, 2005. The entire contents of each of these application is expressly incorporated herein by reference.

### FIELD OF THE INVENTION

The present invention relates to peptide nucleic acid (PNA) probes, PNA probe sets and methods for the analysis of microorganisms optionally present in a sample. Microorganisms targeted by the probes of this invention include Escherichia coli, Klebsiella pneumoniae, Klebsiella oxytoca, Acinetobacter species, Streptococcus agalactiae, as well as the phylum, Fungi. The invention further relates to diagnostic kits comprising such PNA probes.

### BACKGROUND OF THE INVENTION

The diagnosis of infectious diseases is still often based on classical microbiology methodologies, such as culture and biochemical tests for phenotypic markers, and typically takes from 1-2 days up to weeks or even months before final diagnosis is available. In the meantime, patients are often treated empirically based on preliminary test results and clinical symptoms, which often lead to an unnecessary use of antibiotics and its sequelae.

Conventional biochemical methods for the analysis of microorganisms are slow and misidentifications are well known. Rapid and accurate methods for detection, identification and/or quantitation of microorganisms is thus important in order to ensure optimal antibiotic therapy and patient management.

Despite its name, Peptide Nucleic Acid (PNA) is neither a peptide nor a nucleic acid, it is not even an acid. PNA is a non-naturally occurring polyamid that can hybridize to nucleic acid (DNA and RNA) with sequence specificity (see: U.S. Pat. No. 5,539,082) and Egholm et al., Nature 365:566-568 (1993)) according to Watson-Crick base paring rules. However, whereas nucleic acids are biological materials that play a central role in the life of living species as agents of genetic transmission and expression, PNA is a recently developed totally artificial molecule, conceived in the minds of chemists and made using synthetic organic chemistry. PNA also differs structurally from nucleic acid. Although both can employ common nucleobases (A, C, G, T, and U), the backbones of these molecules are structurally diverse. The backbones of RNA and DNA are composed of repeating phosphodiester ribose and 2-deoxyribose units. In contrast, the backbones of the most common PNAs are composed of (aminoethyl)-glycine subunits. Additionally, in PNA the nucleobases are connected to the backbone by an additional methylene carbonyl moiety. PNA is therefore not an acid and therefore contains no charged acidic groups such as those present in DNA and RNA. The non-charged backbone allows PNA probes to hybridize under conditions that are destabilizing to DNA and RNA, attributes that enable PNA probes to access targets, such as highly structured rRNA and double stranded DNA, known to be inaccessible to DNA probes (See: Stephano & Hyldig-Nielsen, IBC Library Series Publication #948. International Business Communication, Southborough, Mass., pp. 19-37 (1997)). PNAs are useful candidates for investigation when developing probe-based hybridization assays because they hybridize to nucleic acids with sequence specificity. PNA probes, however are not the equivalent of nucleic acid probes in structure or function.

Comparative analysis of ribosomal RNA (rRNA) sequences or genomic DNA sequences corresponding to said rRNA (rDNA) has become a widely accepted method for establishing phylogenetic relationships between bacterial species (Woese, Microbiol. Rev. 51:221-271 (1987)), and Bergey's Manual of systematic bacteriology has been revised based on rRNA or rDNA sequence comparisons. Ribosomal RNA or rDNA sequence differences between closely related species enable design of specific probes for microbial identification and thus enable diagnostic microbiology to be based on a single genetic marker rather than a series of phenotypic markers as characterizing traditional microbiology (Delong et al., Science 342:1360-1363 (1989)).

PNA fluorescent in situ hybridization (PNA FISH) technology for detection of specific microorganisms has been described in several articles (see for example Stender et al, J Clin Microbiol. 1999 September; 37(9):2760-5; Rigby et al J Clin Microbiol. 2002 June; 40(6):2182-6; and Oliveira et al J Clin Microbiol. 2002 January; 40(1):247-51.) and several kits are commercially available (AdvanDx, Inc, Woburn Mass.). PNA FISH as its name indicates uses fluorescent labeled PNA probes to illuminate samples based on the specific hybridization of PNA probes to nucleic acid targets in the sample. Usually PNA probes are designed to detect high copy number targets such as ribosomal RNA. The PNA FISH method is accepted as an improvement over traditional microbiological detection techniques in that it has the potential to provide rapid and accurate identification of microorganisms to the species level. One limitation of PNA FISH technology is the availability of proven probe sequences. Since there is some expense required to manufacture and test novel probes, and there is some art to the design of probes, novel probe sequences of proven value are infrequently described.

### SUMMARY OF THE INVENTION

This invention is directed to PNA probes and their use as well as kits useful for the analysis of microorganisms optionally present in a sample of interest. In accordance with the invention, the PNA probes are directed to rRNA or the genomic sequences corresponding to said rRNA (rDNA) or its complement. In preferred embodiments the probes of this invention are used for in situ hybridization analysis of microorganisms optionally present in a sample, most preferably the in situ hybridization analysis is fluorescence in situ hybridization analysis.

In one embodiment, this invention is directed to PNA probes for detection, identification and/or quantitation of microorganisms, e.g., Escherichia coli, Klebsiella pneumoniae, Klebsiella oxytoca, Streptococcus agalactiae, fungi, and Acinetobacter species.

These PNA probes have the inherent physico/chemical characteristics of PNA probes as compared to nucleic acid probes, such that rapid and accurate analysis can be performed using just a single PNA probe. Furthermore, many microorganisms have a relatively rigid cell wall where the improved penetration of PNA probes also offers an advantage as compared to nucleic acid probes when applied in fluorescence in situ hybridization assays. Where nucleic acid probes require fixation and permeabilization with cross-linking agents and/or enzymes (for example see Kempf et al., J. Clin. Microbiol 38:830-838 (2000)), these PNA probes can be applied directly following smear preparation as exemplified in example 1.

In a preferred embodiment, these PNA probes have a relatively short nucleobase sequence, such as 8-17 bases, most preferably they are 15 nucelobases as described in example 1. Naturally occurring nucleic acid probes are typically at least 18 nucleobases (For example see Kempf et al., J. Clin. Microbiol 38:830-838 (2000)) due to their lower Tm values. This difference provides these PNA probes with better discrimination to closely related non-target sequences with only a single or just a few nucleobase difference(s) as required for analysis of rRNA or rDNA from microorganisms.

PNA probe nucleobase sequences according to the invention are selected from the group consisting of: ACA-CAC-ACT-GAT-TCA (SEQ ID NO:1), CAC-CTA-CAC-ACC-AGC (SEQ ID NO:2), CAC-TTA-CCA-TCA-GCG (SEQ ID NO:3), ACA-CCA-AAC-CTC-AGC (SEQ ID NO:4), ACA-CCA-AAC-ATC-AGC (SEQ ID NO:5), CCC-TAG-TCG-GCA-TAG (SEQ ID NO:6), CCA-AGA-GAT-CCG-TTG (SEQ ID NO:7), CCT-CTC-ATC-GCA-TTA-C (SEQ ID NO:8), and GCT-CAC-CAG-TAT-CG (SEQ ID NO:9) One or more of these probes, or the complements thereof, are included in the most preferred probe sets of this invention.

Preferably probes of this invention are labeled with at least one detectable moiety, wherein the detectable moiety or moieties are selected from the group consisting of: a conjugate, a branched detection system, a chromophore, a fluorophore, a spin label, a radioisotope, an enzyme, a hapten, an acridinium ester and a luminescent compound. Fluorescent labeled probes of this invention may be self-reporting, most preferably self-reporting fluorescent probes of this invention are PNA Linear Beacons. Probes of this invention may contain two or more fluorescent labels which are independently detectable, or detectable by coincidental fluorescence.

In other conceptions of the present invention PNA probes are included which contain moieties that add functionality to the probe. Such moieties include but are not limited to spacer and linker groups. Likewise, PNA probes of this invention encompass probes attached to a solid support such as but not limited to a membrane, a slide, an array, a bead, or a particle.

Probe sets of this invention include two or more PNA probes for the analysis of microorganisms optionally present in a sample. Probe sets are preferably labeled with a detectable moiety. Probe sets may be labeled with the same detectable moiety, or they may be differently labeled for independent analysis of probe signals. It is within the conception of this invention that two or more differently labeled fluorescent probes of a probe set may be used to create a third signal by coincidental fluorescence.

The method according to the invention comprises contacting a sample with one or more of the PNA probes described above. According to the method, the presence, absence and/or number of microorganisms in the sample are then detected, identified and/or quantitated and/or the susceptibility to antibiotics is determined by correlating the hybridization, under suitable hybridization conditions, of the probing nucleobase sequence of the probe to the target sequence. Consequently, the analysis is based on a single assay with a definitive outcome. In contrast, current routine methods for analysis of microorganisms are based on multiple phenotypic characteristics involving multiple tests.

In preferred embodiments the methods of this invention are used for in situ hybridization analysis of microorganisms optionally present in a sample, most preferably the in situ hybridization analysis is fluorescence in situ hybridization analysis. In preferred methods of the invention, the sample is a biological sample, including but not limited to blood, urine, secretion, sweat, sputum, stool, mucous, or cultures thereof.

Preferred methods of the invention optionally include non-labeled blocking probes to reduce or eliminate hybridization of PNA probes to non-target sequences. Methods of this invention do not include the use of cross-linking reagents or enzymes prior to hybridization.

The methods of this invention may also be used to detect nucleic acid targets generated, synthesized or amplified in a reaction. Preferred methods for generating, synthesizing or amplifying targets include PCR, LCR, SDA, TMA, RCA and Q-beta replicase.

Methods of the invention include those in which the targets are immobilized to a surface, such as a membrane, a slide, a bead, or a particle and which may furthermore be a component of an array. Optionally, the methods may include PNA probes which are immobilized to a surface such as a membrane, a slide, a bead, or a particle, and may furthermore be a component of an array.

In a highly preferred embodiment of the invention, the medical treatment of a patient includes i.) obtaining a sample from the patient, ii.) determining the presence, amount and/or identity of microorganisms in the sample, and iii.) optionally administering at least one antibiotic compound towards treatment of the infection.

In still another embodiment, this invention is directed to kits suitable for performing an assay that detect, identify and/or quantitate microorganisms optionally present in a sample and/or determination of antibiotic resistance. The kits of this invention comprise one or more PNA probes and other reagents or compositions that are selected to perform an assay or otherwise simplify the performance of an assay. Preferred kit formats include kits designed to perform in situ hybridization assays, and kits designed to perform real-time PCR assays. Preferred kits are designed to examine samples such as clinical specimens, or cultures thereof.

Those of ordinary skill in the art will appreciate that a suitable PNA probe need not have exactly these probing nucleobase sequences to be operative but often modified according to the particular assay conditions. For example, shorter PNA probes can be prepared by truncation of the nucleobase sequence if the stability of the hybrid needs to be modified to thereby lower the Tm and/or adjust for stringency. Similarly, the nucleobase sequence may be truncated at one end and extended at the other end as long as the discriminating nucleobases remain within the sequence of the PNA probe. Such variations of the probing nucleobase sequences within the parameters described herein are considered to be embodiments of this invention.

The PNA probes, methods and kits of this invention have been demonstrated to be both sensitive and specific for the microorganisms they are directed to. Moreover, the assays described herein are rapid (less than 3 hours) and capable of analysis of microorganisms in a single assay.

Those of ordinary skill in the art will also appreciate that the complement probing sequence is equally suitable for assays, such as but not limited to real-time PCR, that are using rDNA as target.

### DETAILED DESCRIPTION OF THE INVENTION

### I. Definitions

a. As used herein, the term “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids.

Non-limiting examples of suitable nucleobases include, but are not limited to: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobase include those nucleobases illustrated in FIGS. 2(A) and 2(B) of Buchardt et al. (U.S. Pat. No. 6,357,163 or WO92/20702 or WO92/20703), herein incorporated by reference).

b. As used herein, the term “nucleobase sequence” means any segment of a polymer that comprises nucleobase-containing subunits. Non-limiting examples of suitable polymers or polymer segments include oligodeoxynucleotides, oligoribonucleotides, peptide nucleic acids, nucleic acid analogs, nucleic acid mimics, and/or chimeras.
c. As used herein, the term “target sequence” means the nucleobase sequence that is to be detected in an assay.
d. As used herein, the term “probe” means a polymer (e.g. a DNA, RNA, PNA, chimera or linked polymer) having a probing nucleobase sequence that is designed to sequence-specifically hybridize to a target sequence of a target molecule of an organism of interest.
e. As used herein, “analyze” means that the individual bacteria are marked for detection, identification and/or quantitation and/or for determination of resistance to antibiotics (antimicrobial susceptibility).
f. As used herein, “peptide nucleic acid” or “PNA” means any oligomer or polymer segment comprising two or more PNA subunits (residues), including, but not limited to, any of the oligomer or polymer segments referred to or claimed as a peptide nucleic acid in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,201,103, 6,350,853, 6,357,163, 6,395,474, 6,414,112, 6,441,130, 6,451,968; 6,969,766, and 7022851 all of which are herein incorporated by reference. The term “peptide nucleic acid” or “PNA” shall also apply to any oligomer or polymer segment comprising two or more subunits of those nucleic acid mimics described in the following publications: Lagriffoul et al., Bioorganic & Medicinal Chemistry Letters, 4:1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al Bioorg. Med. Chem. Lett. 7: 687-690 (1997); Krotz et al., Tett. Lett. 36: 6941-6944 (1995); Lagriffoul et al., Bioorg. Med. Chem. Lett. 4:1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000. In the most preferred embodiment, a PNA subunit consists of a naturally occurring or non-naturally occurring nucleobase attached to the aza nitrogen of the N-[2-(aminoethyl)] glycine backbone through a methylene carbonyl linkage.
g. As used herein, the terms “label” and “detectable moiety” are interchangeable and shall refer to moieties that can be attached to a probe to thereby render the probe detectable by an instrument or method.
h. As used herein, the term “coincidental fluorescence” is used to describe the perception of a color which is generated by the simultaneous detection of light emissions of two or more labels located near enough in space so as to be irresolvable. The detection of coincidental fluorescence can be either by eye or a photon-sensitive device.

### 2. Description

### I. General

### PNA Synthesis:

Methods for the chemical assembly of PNAs are well known (See: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,201,103, 6,350,853, 6,357,163, 6,395,474, 6,414,112, 6,441,130, 6,451,968, 6,969,766, and 7,022,851; all of which are herein incorporated by reference (Also see: PerSeptive Biosystems and/or Applied Biosystems Product Literature)). As a general reference for PNA synthesis methodology also please see: Nielsen et al., Peptide Nucleic Acids; Protocols and Applications, Horizon Scientific Press, Norfolk England (1999).

Chemicals and instrumentation for the support bound automated chemical assembly of peptide nucleic acids are now commercially available. Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkyl amino terminus that is condensed with the next synthon to be added to the growing polymer.

PNA may be synthesized at any scale, from submicromole to millimole, or more. PNA can be conveniently synthesized at the 2 μmole scale, using Fmoc(Bhoc) protecting group monomers on an Expedite Synthesizer (Applied Biosystems) using a XAL, PAL or many other suitable commercially available peptide synthesis supports. Alternatively, the Model 433A Synthesizer (Applied Biosystems) with a suitable solid support (e.g. MBHA support) can be used. Moreover, many other automated synthesizers and synthesis supports can be utilized. Synthesis can be performed using continuous flow method and/or a batch method. PNA can also be manually synthesized.

### PNA Labeling:

Preferred non-limiting methods for labeling PNAs are described in U.S. Pat. Nos. 6,110,676, 6,361,942, 6, 355,421, 6,969,766, and 7,022,851 the examples section of this specification or are otherwise well known in the art of PNA synthesis and peptide synthesis.

### Labels:

Non-limiting examples of detectable moieties (labels) suitable for labeling PNA probes used in the practice of this invention would include a dextran conjugate, a branched nucleic acid detection system, a chromophore, a fluorophore, a spin label, a radioisotope, an enzyme, a hapten, an acridinium ester and a chemiluminescent compound.

Other suitable labeling reagents and preferred methods of attachment would be recognized by those of ordinary skill in the art of PNA, peptide or nucleic acid synthesis.

Preferred haptens include 5 (6)-carboxyfluorescein, 2,4-dinitrophenyl, digoxigenin, and biotin.

Preferred fluorochromes (fluorophores) include 5 (6)-carboxyfluorescein (Flu), 6-((7-amino-4-methylcoumarin-3-acetyl)amino) hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2,3,3,5,5 and 5.5 are available as NHS esters from Amersham, Arlington Heights, Ill.), JOE, Tamara or the Alexa dye series (Molecular Probes, Eugene, Oreg.).

Preferred enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP) and most preferably, soy bean peroxidase (SBP).

### Unlabeled Probes:

The probes that are used for the practice of this invention need not be labeled with a detectable moiety to be operable within the methods of this invention, for example when attached to a solid support

### Self-Indicating Probes:

Beacon probes are examples of self-indicating probes which include a donor moiety and a acceptor moiety. The donor and acceptor moieties operate such that the acceptor moieties accept energy transferred from the donor moieties or otherwise quench signal from the donor moiety. Though the previously listed fluorophores (with suitable spectral properties) might also operate as energy transfer acceptors, preferably, the acceptor moiety is a quencher moiety. Preferably, the quencher moiety is a non-fluorescent aromatic or heteroaromatic moiety. The preferred quencher moiety is 4-((4-(dimethylamino) phenyl) azo) benzoic acid (dabcyl). In a preferred embodiment, the self-indicating Beacon probe is a PNA Linear Beacon as more fully described in U.S. Pat. No. 6,485,901.

In another embodiment, the self-indicating probes of this invention are of the type described in WIPO patent application WO97/45539. These self-indicating probes differ as compared with Beacon probes primarily in that the reporter must interact with the nucleic acid to produce signal.

### Spacer/Linker Moieties:

Generally, spacers are used to minimize the adverse effects that bulky labeling reagents might have on hybridization properties of probes. Preferred spacer/linker moieties for the nucleobase polymers of this invention consist of one or more aminoalkyl carboxylic acids (e.g. aminocaproic acid), the side chain of an amino acid (e.g. the side chain of lysine or ornithine), natural amino acids (e.g. glycine), aminooxyalkylacids (e.g. 8-amino-3,& dioxaoctanoic acid), alkyl diacids (e.g. succinic acid), alkyloxy diacids (e.g. diglycolic acid) or alkyldiamines (e.g. 1,8-diamino-3,6-dioxaoctane).

### Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

### Suitable Hybridization Conditions:

Generally, the more closely related the background causing nucleic acid sequences are to the target sequence, the more carefully stringency must be controlled. Blocking probes may also be used as a means to improve discrimination beyond the limits possible by optimization of stringency factors. Suitable hybridization conditions will thus comprise conditions under which the desired degree of discrimination is achieved such that an assay generates an accurate (within the tolerance desired for the assay) and reproducible result. The use of PNA probes presents a notable advantage of the invention over conventional methods using DNA probes. Part of the advantage of PNA is that it hybridizes under conditions which are non-preferred or destabilizing to DNA probes. Examples of such conditions include conditions of low ionic strength (low salt), and high concentrations of lower alcohols, i.e, 50% ethanol.

Aided by no more than routine experimentation and the disclosure provided herein, those of skill in the art will easily be able to determine suitable hybridization conditions for performing assays utilizing the methods and compositions described herein. Suitable in situ hybridization or PCR conditions comprise conditions suitable for performing an in situ hybridization or PCR procedure. Thus, suitable in situ hybridization or PCR conditions will become apparent to those of skill in the art using the disclosure provided herein, with or without additional routine experimentation.

### Blocking Probes:

Blocking probes are nucleic acid or non-nucleic acid probes that can be used to suppress the binding of the probing nucleobase sequence of the probing polymer to a non-target sequence. Preferred blocking probes are PNA probes (see: U.S. Pat. No. 6,110,676). It is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence. Thus, blocking probes can be used with the methods, kits and compositions of this invention to suppress the binding of the probes to a non-target sequence that might be present and interfere with the performance of the assay. Blocking probes are particularly advantageous for discrimination to the phylogenetically closest related species.

### Probing Nucleobase Sequence:

The probing nucleobase sequence of a probe of this invention is the specific sequence recognition portion of the construct. Therefore, the probing nucleobase sequence is a nucleobase sequence designed to hybridize to a specific target sequence wherein the presence, absence or amount of the target sequence can be used to directly or indirectly detect the presence, absence or number of organisms of interest in a sample. Consequently, with due consideration to the requirements of a probe for the assay format chosen, the length and sequence composition of the probing nucleobase sequence of the probe will generally be chosen such that a stable complex is formed with the target sequence under suitable hybridization conditions.

The preferred probing nucleobase sequence of the probe of this invention that is suitable for the detection, identification and/or enumeration of Escherichia coli comprises a nucleobase sequence of: ACA-CAC-ACT-GAT-TCA (SEQ ID NO: 1) and the complement thereto.

The preferred probing nucleobase sequence of the probes of this invention that are suitable for the detection, identification and/or enumeration of Streptococcus agalactiae comprise the nucleobases sequence of: ACA-CCA-AAC-CTC-AGC (SEQ ID NO: 4), ACA-CCA-AAC-ATC-AGC (SEQ ID NO:SEQ ID NO: 5), and the complements thereto. Probes designed based on SEQ ID NO:SEQ ID NO: 4 and SEQ ID NO:SEQ ID NO: 5 differ by only one base pair, are used to detect naturally occurring variants of the Streptococcus agalactiae (Kempf et al 2003).

The preferred probing nucleobase sequences of the probes of this invention that are suitable for the detection, identification and/or enumeration of Klebsiella pneumoniae comprise a nucleobase sequence of: CAC-CTA-CAC-ACC-AGC (SEQ ID NO: 2), and the complement thereto; and for the detection, identification and/or enumeration of Klebsiella oxytoca, comprise a nucleobase sequence of: CAC-TTA-CCA-TCA-GCG (SEQ ID NO: 3), and the complement thereto.

The preferred probing nucleobase sequence of the probes of this invention that are suitable for the detection, identification and/or enumeration of fungi comprise a nucleobase sequence of: CCC-TAG-TCG-GCA-TAG (SEQ ID NO: 6), CCA-AGA-GAT-CCG-TTG (SEQ ID NO: 7), and the complements thereto. Seq Id Nos. 6 and 7 correspond to a pan-fungal probes which correspond to 18S and 5.8S rRNA sequences respectively. These pan-fungal probes are predicted to detect a wide range of fungal families by sequence alignment. The pan-fungal probes are predicted to have particularly wide coverage of species of fungi in the phyla ascomycota and basidiomycota.

The preferred probing nucleobase sequences of the probes of this invention that are suitable for the detection, identification and/or enumeration of Acinetobacter species comprise nucleobase sequences of: CCT-CTC-ATC-GCA-TTA-C (SEQ ID NO:8), and GCT-CAC-CAG-TAT-CG (SEQ ID NO:9), and the complements thereto.

This invention contemplates that variations in these identified probing nucleobase sequences shall also provide probes that are suitable for the analysis of microorganisms. Variations of the probing nucleobase sequences within the parameters described herein are considered to be an embodiment of this invention.

Common variations include, deletions, insertions and frame shifts. Additionally, a shorter probing nucleobase sequence can be generated by truncation of the sequence identified above.

A probe of this invention will generally have a probing nucleobase sequence that is exactly complementary to the target sequence. Alternatively, a substantially complementary probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15: 331-335 (1997)). Consequently, the probing nucleobase sequence may be only 90% homologous to the probing nucleobase sequences identified above. Substantially complementary probing nucleobase sequence within the parameters described above are considered to be an embodiment of this invention.

Complements of the probing nucleobase sequence are considered to be an embodiment of this invention, since it is possible to generate a suitable probe if the target sequence to be detected has been amplified or copied to thereby generate the complement to the identified target sequence.

Detection, Identification and/or Enumeration:

By detection is meant analysis for the presence or absence of the organism optionally present in the sample. By identification is meant establishment of the identity of the organism by genus and species name. By quantitation is meant enumeration of the organisms in a sample. Some assay formats provide simultaneous detection, identification and enumeration (for example see Stender, H. et al., J. Microbiol. Methods. 45:31-39 (2001), others provide detection and identification (for example see Stender, H. et al., Int. J. Tuberc. Lung Dis. 3:830-837 (1999) and yet other assay formats just provide identification (for example see Oliveira, K et al. J. Clin. Microbiol. 40:247-251 (2002)).

### Antibiotic Resistance

By determination of resistance to antibiotics is meant analysis of an organisms susceptibility to antibiotics based on specific genes or mutations associated with resistance or susceptibility to antimicrobial agents.

### II. Preferred Embodiments of the Invention

a. PNA Probes:

In one embodiment, the PNA probes of this invention are suitable for detecting, identifying and/or quantitating microroganisms. General characteristics (e.g. length, labels, nucleobase sequences, linkers etc.) of PNA probes suitable for the analysis have been previously described herein. The preferred probing nucleobase sequence of PNA probes of this invention are listed in Table 1.

TABLE 1

| | |
|---|---|
| Sequence ID | Nucleobase sequence |

| | |
|---|---|
| SEQ ID NO: 1 | ACA-CAC-ACT-GAT-TCA |
| SEQ ID NO: 2 | CAC-CTA-CAC-ACC-AGC |
| SEQ ID NO: 3 | CAC-TTA-CCA-TCA-GCG |
| SEQ ID NO: 4 | ACA-CCA-AAC-CTC-AGC |
| SEQ ID NO: 5 | ACA-CCA-AAC-ATC-AGC |
| SEQ ID NO: 6 | CCC-TAG-TCG-GCA-TAG |
| SEQ ID NO: 7 | CCA-AGA-GAT-CCG-TTG |
| SEQ ID NO: 8 | CCT-CTC-ATC-GCA-TTA-C |
| SEQ ID NO: 9 | GCT-CAC-CAG-TAT-CG |

The PNA probes of this invention may comprise only a probing nucleobase sequence (as previously described herein) or may comprise additional moieties. Non-limiting examples of additional moieties include detectable moieties (labels), linkers, spacers, natural or non-natural amino acids, or other subunits of PNA, DNA or RNA. Additional moieties may be functional or non-functional in an assay. Generally however, additional moieties will be selected to be functional within the design of the assay in which the PNA probe is to be used. The preferred PNA probes of this invention are labeled with one or more detectable moieties selected from the group consisting of fluorophores, enzymes and haptens.

In preferred embodiments, the probes of this invention are used in in situ hybridization (ISH) and fluorescence in situ hybridization (FISH) assays. Excess probe used in an ISH or FISH assay typically must be removed so that the detectable moiety of the specifically bound probe can be detected above the background signal that results from still present but unhybridized probe. Generally, the excess probe is washed away after the sample has been incubated with probe for a period of time. However, the use of self-indicating probes is a preferred embodiment of this invention, since there is no requirement that excess self-indicating probe be completely removed (washed away) from the sample since it generates little or no detectable background. In addition to ISH or FISH assays, self-indicating probes comprising the selected probing nucleobase sequence described herein are particularly useful in all kinds of homogeneous assays such as in real-time PCR or useful with self-indicating devices (e.g. lateral flow assay) or self-indicating arrays.

b. PNA Probe Sets

Probe sets of this invention comprise two of more PNA probes. In one embodiment, some of the PNA probes of the set can be blocking probes. Probes sets may include any group of two or more of the probes of this invention, whether labeled or non-labeled, and may also include probes not specifically described here, but which include at least one of the probes of this invention. Preferred probes sets include CAC-CTA-CAC-ACC-AGC (SEQ ID NO:2) and CAC-TTA-CCA-TCA-GCG (SEQ ID NO:3) for detection of Klebsiella species; ACA-CCA-AAC-CTC-AGC (SEQ ID NO:4), and ACA-CCA-AAC-ATC-AGC (SEQ ID NO:5) for detection of Streptococcus agalactiae variants; CCC-TAG-TCG-GCA-TAG (SEQ ID NO:6) and CCA-AGA-GAT-CCG-TTG (SEQ ID NO:7) for detection of fungi, and CCT-CTC-ATC-GCA-TTA-C (SEQ ID NO:8), and GCT-CAC-CAG-TAT-CG (SEQ ID NO:9) for detection of Acinetobacter species.

c. Methods:

In another embodiment, this invention is directed to a method suitable for analysis of microorganisms optionally in a sample. The general and specific characteristics of PNA probes suitable for the analysis of microorganisms have been previously described herein. Preferred probing nucleobase sequences are listed in Table 1.

The method for analysis of microorganisms in a sample comprises contacting the sample with one or more PNA probes suitable for hybridization to a target sequence which is specific.

According to the method, the microorganism in the sample is then detected, identified and/or quantitated or its resistance to antibiotics is determined. This is made possible by correlating hybridization, under suitable hybridization conditions, of the probing nucleobase sequence of a PNA probe to the target sequence of microorganism sought to be detected with the presence, absence or number of the microorganisms in the sample. Typically, this correlation is made possible by direct or indirect detection of the probe/target sequence hybrid.

### Fluorescence in Situ Hybridization and Real-Time PCR:

The PNA probes, methods, kits and compositions of this invention are particularly useful for the rapid probe-based analysis of microorganisms. In preferred embodiments, in situ hybridization or PCR is used as the assay format for analysis of microorganisms. Most preferably, fluorescence in situ hybridization (PNA FISH) or real-time PCR is the assay format. (Reviewed by Stender et al. J. Microbiol. Methods 48:1-17 (2002)). Preferably, smears for PNA FISH analysis are not treated with cross-linking agents or enzymes prior to hybridization.

### Exemplary Assay Formats:

Exemplary methods for performing PNA FISH can be found in: Oliveira et., J. Clin. Microbiol 40:247-251 (2002), Rigby et al., J. Clin. Microbiol. 40:2182-2186 (2002), Stender et al., J. Clin. Microbiol. 37:2760-2765 (1999), Perry-O'Keefe et al., J. Microbiol. Methods 47:281-292 (2001). According to one method, a smear of the sample, such as, but not limited to, a positive blood culture, is prepared on microscope slides and covered with one drop of the fluorescein-labeled PNA probe in hybridization buffer. A coverslip is placed on the smear to ensure an even coverage, and the slide is subsequently placed on a slide warmer or incubator at 55° C. for 90 minutes. Following hybridization, the coverslip is removed by submerging the slide into a pre-warmed stringent wash solution and the slide is washed for 30 minutes. The smear is finally mounted with one drop of mounting fluid, covered with a coverslip and examined by fluorescence microscopy.

Microorganisms optimally present in a sample which may be analyzed with the PNA probes contained in the kits of this invention can be determined by several instruments, such as but not limited to the following examples: microscope (for example see Oliveira et al., J. Clin. Microbiol 40:247-251 (2002)), film (for example see Perry-O'Keefe et al., J. Appl. Microbiol. 90:180-189) (2001), camera and instant film (for example see Stender et al., J. Microbiol. Methods 42:245-253 (2000)), luminometer (for example see Stender et al., J. Microbiol. Methods. 46:69-75 (2001), laser scanning device (for example see Stender et al., J. Microbiol. Methods. 45: 31-39 (2001) or flow cytometer (for example see Wordon et al., Appl. Environ. Microbiol. 66:284-289 (2000)). Automated slide scanners and flow cytometers are particularly useful for rapidly quantitating the number of microorganisms present in a sample of interest.

Exemplary methods for performing real-time PCR using self-reporting PNA probes can be found in: Fiandaca et al., Abstract, Nucleic Acid-Based technologies. DNA/RNA/PNA Diagnostics, Washington, D.C., May 14-16, 2001, and Perry-O'Keefe et al., Abstract, International Conference on Emerging Infectious Diseases, Atlanta, 2002.

d. Kits:

In yet another embodiment, this invention is directed to kits suitable for performing an assay, which analyses microorganisms optionally present in a sample. The general and preferred characteristics of PNA probes suitable for the analysis of microorganisms have been previously described herein. Preferred probing nucleobase sequences are listed in Table 1. Furthermore, methods suitable for using the PNA probes to analyze microorganisms in a sample have been previously described herein.

The kits of this invention comprise one or more PNA probes and other reagents or compositions which are selected to perform an assay or otherwise simplify the performance of an assay used to analyze microorganisms in a sample.

The kits can, for example, be used for in situ assays or for use with nucleic acid amplification technologies Non-limiting examples of nucleic acid amplification technologies include, but are not limited to, Polymerase Chain Reaction (PCR), Ligase Chain Reaction (LCR), Strand Displacement Amplification (SDA), Transcription-Mediated Amplification (TMA), Q-beta replicase amplification (Q-beta) and Rolling Circle Amplification (RCA).

Accordingly, in some embodiments the other reagents can comprise, buffers, enzymes and/or master mixes for performing an in situ or nucleic acid amplification based assay.

e. Exemplary Applications for Using the Invention:

The PNA probes, methods and kits of this invention are particularly useful for the analysis of microorganisms in clinical samples, e.g. urine, blood, wounds, sputum, laryngeal swabs, gastric lavage, bronchial washings, biopsies, aspirates, expectorates as well as in food, beverages, water, pharmaceutical products, personal care products, dairy products or environmental samples and cultures thereof.

### Additional Detection Strategies

Though probes labeled with fluorescein and tamra are described, it is within the concept of this invention that any combination of fluorescent labels could be used which produce a perceivable third color. Likewise, use of two or more labels to produce multiple perceivable colors is also envisioned. Potential fluorescent labels are included in the description. Coincidental fluorescence of two or more fluorescent moieties has been demonstrated to be useful in the generation of a spectrum of colors (Kool et al JACS 2003). Combination colors are made through “mixtures” of two or more fluorophores, and adjustment of their ratios. For example, a combination of two parts red and one part green produces a different color that one part red and two parts green. Though accurate discrimination of these various shades by eye may have a practical limit, it is not difficult to conceive of a device which could accurately perceive such subtle color variations.

Detectable and Independently Detectable Moieties/Multiplex Analysis: A multiplex hybridization assay can be performed in accordance with this invention. In a multiplex assay, numerous conditions of interest can be simultaneously examined.

Multiplex analysis relies on the ability to sort sample components or the data associated therewith, during or after the assay is completed. In preferred embodiments of the invention, one or more distinct independently detectable moieties can be used to label two or more different probes used in an assay. The ability to differentiate between and/or quantitate each of the independently detectable moieties provides the means to multiplex a hybridization assay. Correlation of the hybridization of each of the distinctly (independently) labeled probes to particular nucleic acid sequences is indicative of presence, absence or quantity of each organism sought to be detected in the sample.

Consequently, the multiplex assays of this invention can be used to simultaneously detect the presence, absence or quantity of two or more different organisms (e.g. species of Klebsiella) in the same sample and in the same assay. For example, a multiplex assay may utilize two or more PNA probes, each being labeled with an independently detectable fluorophore, or a set of independently detectable fluorophores.

Accordingly, the invention provides for a method to treat a patient which in embodiment includes at least one of and preferably all of the following steps:
a) obtaining a biological sample from the patient
b) determining the presence, amount and/or identity of microorganisms; and
c) administering at least one antibiotic with activity towards elimination of the infection.

The invention further provides for a PNA probe set that includes at least one of the PNA probes provided herein, preferably two or more probes, wherein the probes to make a third color by coincidental fluorescence.

Having described the preferred embodiments of the invention, it will now become apparent to one of skill in the art that other embodiments incorporating the concepts described herein may be used. It is felt, therefore, that these embodiments should not be limited to disclosed embodiments but rather should be limited only by the spirit and scope of the following claims.

### EXAMPLES

This invention is now illustrated by the following example, which are not intended to be limiting in any way.

### Example 1

### PNA Probe Sequence

| | |
|---|---|
| Kpn23S01-Flu | |
| Flu-OO-CACCTACACACCAGC-NH₂ | (SEQ ID NO: 2) |
| Kox23S01-Tam | |
| Tam-OO-CACTTACCATCAGCG-NH₂ | (SEQ ID NO: 3) |

(Note: Conventional nomenclature used to illustrate the termini of the PNA probe; O=8-amino-3,6-dioxaoctanoic acids; flu=5(6)-carboxy-fluorescein, tam=5(6)-carboxytetramethyrhodamine)

### Bacterium Strains

Overnight cultures of reference strains were prepared (American Type Culture Collection, Manassas, Va.) representing various Gram negative rods, including Klebsiella pneumoniae, and Klebsiella oxytoca species.

### Preparation of Smears.

For each strain, smears were prepared on a 8-mm diameter well of a teflon-coated microscope slide (AdvanDx, Woburn, Mass.) by mixing one drop of culture with one drop of phosphate-buffered saline containing 1% (v/v) Triton X-100. The slide was then placed on a 55° C. slide warmer for 20 min at which point the smears were dry. Subsequently, the smears were disinfected by immersion into 96% (v/v) ethanol for 5-10 minutes and air-dried.

### Fluorescence in Situ Hybridization (FISH).

Smears were covered with a drop of hybridization solution containing 10% (w/v) dextran sulfate, 10 mM NaCl, 30% (v/v) formamide, 0.1% (w/v) sodium pyrophosphate, 0.2% (w/v) polyvinylpyrrolidone, 0.2% (w/v) ficoll, 5 mM Na₂EDTA, 1% (v/v) Triton X-100, 50 mM Tris/HCl pH 7.5 and 500 nM Kpn23S01-Flu and 500 nM Kox23S01-Tam probes (Flu is a fluorescent dye which produces a characteristic green fluorescence, Tam is a fluorescent dye which produces a characteristic red fluorescence). Coverslips were placed on the smears to ensure even coverage with hybridization solution, and the slides were subsequently placed on a slide warmer (Slidemoat, Boekel, Germany) and incubated for 90 min at 55° C. Following hybridization, the coverslips were removed by submerging the slides into approximately 20 ml/slide pre-warmed 25 mM Tris, pH 10, 137 mM NaCl, 3 mM KCl in a water bath at 55° C. and washed for 30 min. Each smear was finally mounted using one drop of Mounting medium (AdvanDx, Woburn, Mass.) and covered with a coverslip. Microscopic examination was conducted using a fluorescence microscope equipped with a FITC/Texas Red dual band filter set. Klebsiella pneumoniae, was identified by green fluorescent rods and Klebsiella oxytoca was identified by red fluorescent rods. Results are recorded in Table 2.

TABLE 2

| Organism | Result |
|---|---|
| Escherichia coli | Negative |
| Klebsiella pneumoniae | Positive/Green |
| Klebsiella oxytoca | Positive/Red |
| Pseudomonas aeruginosa | Negative |
| Pseudomonas florescens | Negative |
| Pseudomonas mendocina | Negative |
| Pseudomonas mucidolens | Negative |
| Pseudomonas pseudoalcigenes | Negative |
| Pseudomonas putida | Negative |
| Pseudomonas syringe | Negative |

With reference to Table 2, only two of the ten Gram-negative rod species tested gave a positive result, the Klebsiella pneumoniae sample contained green fluorescent rods, and the Klebsiella oxytoca sample contained red fluorescent rods. The positive results demonstrate that the probe mixture tested produces species specific signals of green and red with Klebsiella pneumoniae and Klebsiella oxytoca respectively.

### Example 2

### PNA Probe Sequence

| | |
|---|---|
| Saga16S03-Flu | |
| Flu-OO-ACACCAAACCTCAGC | (Seq. Id. No. 4) |
| Saga16S04-Flu | |
| Flu-OO-ACACCAAACATCAGC | (Seq. Id. No. 5) |

(Note: Conventional nomenclature used to illustrate the termini of the PNA probe; O=8-amino-3,6-dioxaoctanoic acids; flu=5(6)-carboxy-fluorescein)

### Bacterium Strains

Overnight cultures of reference strains were prepared (American Type Culture Collection, Manassas, Va.) representing various Gram positive cocci, including Streptococcus agalactiae.

### Preparation of Smears.

Smears were prepared as described in Example 1.

### Fluorescence in Situ Hybridization (FISH).

Hybridization was performed as described In Example 1 except the probes used were either Saga16S03-Flu at 500 nM, Saga16S04-Flu at 500 nM, or a combination of Saga16S03-Flu and Saga16S04-Flu, both at 500 nM (“dual probe”). Microscopic examination was conducted using a fluorescence microscope equipped with a FITC/Texas Red dual band filter set. Streptococcus agalactiae was identified by green fluorescent cocci. Results are recorded in Table 3.

TABLE 3

| Organism | Saga16S03 | Saga16S04 | Dual Probe |
|---|---|---|---|
| Streptococcus agalactiae | Positive/Green | Negative | Positive/Green |
| Streptococcus equisimilis | Negative | Negative | Negative |
| Streptococcus pyogenes | Negative | Negative | Negative |
| Enterococcus durans | Negative | Negative | Negative |
| Enterococcus faecalis | Negative | Negative | Negative |
| Enterococcus facium | Negative | Negative | Negative |
| Enterococcus hirae | Negative | Negative | Negative |

With reference to Table 3, positive results occurred when either the Saga16S03 probe, or the dual probe set were used to detect S. agalactiae, all other results were negative indicating that the probes/probe sets are specific.

### Example 3

### PNA Probe Sequence

| | | |
|---|---|---|
| PF-Adx1 | Flu-OO-CCCTAGTCGGCATAG | (Seq. Id. No. 6) |
| PF-Adx2 | Flu-OO-CCAAGAGATCCGTTG | (Seq. Id. No. 7) |

(Note: Conventional nomenclature used to illustrate the termini of the PNA probe; O=8-amino-3,6-dioxaoctanoic acids; flu=5(6)-carboxy-fluorescein)

### Bacteria/Fungal Strains

Overnight cultures of reference strains were prepared (American Type Culture Collection, Manassas, Va.) representing various Gram positive cocci, including Streptococcus agalactiae.

### Preparation of Smears.

Smears were prepared as described in Example 1.

### Fluorescence in Situ Hybridization (FISH).

Hybridization was performed as described in Example 1 except the probes used were either PF-Adx1 at 500 nM, or PF-Adx2 at 500 nM. Microscopic examination was conducted using a fluorescence microscope equipped with a FITC/Texas Red dual band filter set. Fungi was identified by green fluorescent buds or hyphae. Results are recorded in Table 4.

TABLE 4

| Organism | PF-Adx1 | PF-Adx2 |
|---|---|---|
| Candida albicans | Positive/Green | Positive/Green |
| Candida catenulata | Positive/Green | Positive/Green |
| Candida glabrata | Positive/Green | Negative |
| S. aureus | Negative | Negative |
| E. coli | Negative | Negative |
| Saccharomyces cerevisiae | Positive/Green | Positive/Green |

With reference to Table 4, all of the fungal organisms tested with the PF-Adx1 probe including C. albicans, C. catenulata, C. glabrata, and S. cerevisiae were positive. Both of the bacteria species tested (S. aureus and E. coli) were negative. The PF-Adx2 probe produced a similar pattern of results to PF-Adx1, with the exception of C. glabrata, which tested negative. Sequence alignment of published C. glabrata 5.8S rRNA genes demonstrated that there is a single base mismatch in the gene which is the probable cause of the negative result.

### Example 4

### PNA Probe Sequence

| | |
|---|---|
| Eco23S277-Flu | |
| Flu-OO-ACA-CAC-ACT-GAT-TCA | (Seq. Id. No. 1) |
| Abau23S-03b-Flu | |
| Flu-OO-CCT-CTC-ATC-GCA-TTA-C | (Seq. Id. No. 8) |
| Abau16S-04c-Flu | |
| Flu-OO-GCT-CAC-CAG-TAT-CG | (Seq. Id. No. 9) |

(Note: Conventional nomenclature used to illustrate the termini of the PNA probe; O=8-amino-3,6-dioxaoctanoic acids; flu=5(6)-carboxy-fluorescein)

### Bacterium Strains

Overnight cultures of reference strains were prepared (American Type Culture Collection, Manassas, Va.) representing various bacterial species all of which were Gram-negative rods (bacilli).

### Preparation of Smears.

Smears were prepared as described in Example 1.

### Fluorescence in Situ Hybridization (FISH).

Hybridization was performed as described in Example 1 except the probes used were either Eco23S277-Flu at 500 nM, Abau23S-03b-Flu at 200 nM, or Abau16S-04c-Flu at 200 nM. Microscopic examination was conducted using a fluorescence microscope equipped with a FITC/Texas Red dual band filter set. Green fluorescent rods (bacilli) were identified as positives. Results are recorded in Table 4.

TABLE 4

| Organism (ATCC#) | Eco23S277 | Abau16S-04c | Abau23S-03b |
|---|---|---|---|
| Escherichia coli | Positive/Green | Negative | Negative |
| (35218) | | | |
| Escherichia coli | Positive/Green | Not done | Not done |
| (0157:H7) (43888) | | | |
| Acinetobacter | Negative | Positive/Green | Positive/Green |
| calcoaceticus (14987) | | | |
| Acinetobacter | Not done | Positive/Green | Positive/Green |
| baumannii (19606) | | | |
| Pseudomonas | Negative | Negative | Negative |
| aeruginosa (10145) | | | |
| Klebsiella | Negative | Negative | Negative |
| pneumoniae (10031) | | | |
| Klebsiella oxytoca | Negative | Negative | Negative |
| (43086) | | | |

With reference to Table 4, positive results were recorded for the Eco23S277-Flu probe tested against Escherichia coli isolates, and for both the Abau23S-03b-Flu and Abau23S-03b-Flu probes tested against Acinetobacter species, all other results were negative indicating that the probes/probe sets are specific.

### EQUIVALENTS

While this invention has been particularly shown and described with references to preferred embodiments thereof, it will be understood by those of ordinary skill in the art that various changes in form and details may be made therein without departing from the spirit and scope of the invention as defined by the appended claims. Those skilled in the art will be able to ascertain, using no more than routine experimentation, many equivalents to the specific embodiments of the invention described herein. Such equivalents are intended to be encompassed in the scope of the claims.

The disclosures of all references mentioned herein are incorporated by reference.

We claim:

1. A PNA probe comprising a nucleobase sequence suitable for the analysis of microorganisms said PNA probe being selected from the group consisting of SEQ ID NO:1, 2, 3, 4, 5, 6, 7, 8 or 9, and complements thereof.

2. The PNA probe of claim 1, wherein at least a portion of the probe is at least about 86% identical to the nucleobase sequence selected from the group consisting of SEQ ID NO:1, 2, 3, 4, 5, 6, 7, 8 and 9.

3. The PNA probe of claim 2, wherein the probe sequence is 8-18 nucleobases in length.

4. A PNA probe for the detection, identification or quantification of Escherichia coli having the sequence set forth as (SEQ ID NO:1) or a complement thereof.

5. A PNA probe for the detection, identification or quantification of Klebsiella pneumoniae having the sequence set forth as SEQ ID NO:2, or a complement thereof.

6. A PNA probe for the detection, identification or quantification of Klebsiella oxytoca having the sequence set forth as SEQ ID NO:3 or a complement thereof.

7. A PNA probe for the detection, identification or quantification of Streptococcus agalactiae selected from the group consisting of SEQ ID NO:4, SEQ ID NO:5 and complements thereof.

8. A PNA probe for the detection, identification and/or quantification of fungi selected from the group consisting of SEQ ID NO:6, SEQ ID NO:7 and complements thereof.

9. A PNA probe for the detection, identification and/or quantification of Acinetobacter species selected from the group consisting of SEQ ID NO:8, SEQ ID NO:9 and complements thereof.

10. The PNA probe of claim 1, wherein the probe is labeled with at least one detectable moiety.

11. The PNA probe of claim 10, wherein the detectable moiety or moieties are selected from the group consisting of: a conjugate, a branched detection system, a chromophore, a fluorophore, a spin label, a radioisotope, an enzyme, a hapten, an acridinium ester and a luminescent compound.

12. The PNA probe of claim 10, wherein the probe is self-reporting.

13. The PNA probe of claim 12, wherein the probe is a PNA Linear Beacon.

14. The PNA probe of claim 1, wherein the probe is unlabeled.

15. The PNA probe of claim 1, wherein the probe is bound to a support.

16. The PNA probe of claim 15, wherein the probe further comprises a spacer or a linker.

17. The PNA probe of claim 1, wherein in situ hybridization is used for analysis of microorganisms optionally present in the sample.

18. The PNA probe set of claim 1, wherein the probes are differently labeled for independent analysis.

19. A method for the detection, identification or quantitation of microorganisms in a sample, said method comprising:
contacting at least one PNA probe of claim 1 to the sample;
hybridizing the PNA probe to a target sequence of microorganisms in the sample; and
detecting the hybridization as being indicative of presence, identity and/or amount of microorganisms in the sample.

20. A method according to claim 19, wherein the analysis takes place in situ.

21. A method according to claim 20, wherein the analysis takes place by fluorescence in situ hybridization.

22. A method according to claim 21, wherein the method does not involve use of cross-linking reagents or enzymes prior to hybridization.

23. The method of claim 21, wherein the method is used to detect a nucleic acid comprising a target sequence wherein said nucleic acid has been synthesized or amplified in a reaction.

24. The method of claim 23 wherein preferred nucleic acid synthesis or nucleic acid amplification reactions are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase.

25. The method of claim 21, wherein the method further comprises adding at least one blocking probe to reduce or eliminate any hybridization of the PNA probe to non-target sequence.

26. The method of claim 25, wherein the target sequence is immobilized to a surface.

27. The method of claim 21, wherein said PNA probe is immobilized to a surface.

28. The method of claim 27, wherein said PNA probe is one component of an array.

29. The method of claim 21, wherein the method comprises the use of a PNA probe set of claim 17-19.

30. The method of claim 21, wherein the sample is a biological sample.

31. The method of claim 30, wherein the biological sample is blood, urine, secretion, sweat, sputum, stool, mucous, or cultures thereof.

32. A kit for the detection, identification or quantitation of one or more microorganisms comprising one or more PNA probes set forth in claim 1 and instructions for use.

33. The kit of claim 32, wherein the kit is used in an in situ hybridization assay.

34. The kit of claim 32, wherein the kit is used for a real-time PCR assay.

35. The kit of claim 32, wherein the kit is used to examine clinical samples such as clinical specimens or cultures thereof.

36. The PNA probe set comprising at least one PNA probe of claim 1, wherein two or more probes are used to create a third color by coincidental fluorescence.


SEQUENCE LISTING (9 sequences)

SEQ ID NO: 1 - DNA, 15 bp, Artificial Sequence
  Description of Artificial Sequence Synthetic probe
acacacactgattca

SEQ ID NO: 2 - DNA, 15 bp, Artificial Sequence
  Description of Artificial Sequence Synthetic probe
cacctacacaccagc

SEQ ID NO: 3 - DNA, 15 bp, Artificial Sequence
  Description of Artificial Sequence Synthetic probe
cacttaccatcagcg

SEQ ID NO: 4 - DNA, 15 bp, Artificial Sequence
  Description of Artificial Sequence Synthetic probe
acaccaaacctcagc

SEQ ID NO: 5 - DNA, 15 bp, Artificial Sequence
  Description of Artificial Sequence Synthetic probe
acaccaaacatcagc

SEQ ID NO: 6 - DNA, 15 bp, Artificial Sequence
  Description of Artificial Sequence Synthetic probe
ccctagtcggcatag

SEQ ID NO: 7 - DNA, 15 bp, Artificial Sequence
  Description of Artificial Sequence Synthetic probe
ccaagagatccgttg

SEQ ID NO: 8 - DNA, 16 bp, Artificial Sequence
  Description of Artificial Sequence Synthetic probe
cctctcatcgcattac

SEQ ID NO: 9 - DNA, 14 bp, Artificial Sequence
  Description of Artificial Sequence Synthetic probe
gctcaccagtatcg